(19) United States
(12) Patent Application Publication
(10) Pub. No.: US 2014/0221693 A1
(43) Pub. Date: Aug. 7, 2014

(54) Organics Recovery From The Aqueous Phase Of Biomass Catalytic Pyrolysis, And Upgrading Therof

(71) Applicant: KiOR, Inc., Pasadena, TX (US)

(72) Inventors: Maria Magdalena Ramirez-Corredores, Houston, TX (US); Leslie May, Houston, TX (US); Christopher Paradise, La Porte, TX (US); Nicholas Kent, Houston, TX (US); Xiaowei Tong, Houston, TX (US); Rocio Maria Banda, Houston, TX (US); Royce Roemisch, Houston, TX (US)

(21) Appl. No.: 14/090,801

(22) Filed: Nov. 26, 2013

Related U.S. Application Data
(63) Continuation-in-part of application No. 13/762,104, filed on Feb. 7, 2013.

(51) Int. Cl. C07C 45/79; C07C 7/12; C07C 37/82

(52) U.S. Cl. CPC C07C 45/79; C07C 37/82; C07C 7/12
USPC 568/366

(57) ABSTRACT

Disclosed is a process for recovering a water-soluble complex mixture of organic compounds from an aqueous stream through extraction and/or through contact of the aqueous stream with a sorbent or sorbents selected from the group consisting of polymeric microreticular sorbent resins, zeolite-based adsorbents, clay-based adsorbents, activated carbon-based sorbents, and mixtures thereof; and including methods to recover and upgrade the removed organic compounds.

### CROSS REFERENCE TO RELATED APPLICATIONS/INCORPORATION BY REFERENCE STATEMENT

This application is a Continuation-in-Part of, and claims the benefit of, co-pending U.S. application Ser. No. 13/762,104, filed Feb. 7, 2013, which is hereby incorporated by reference in its entirety herein. The entirety of the following patent application is hereby expressly incorporated herein by reference: U.S. patent application Ser. No. 13/212,861 filed on Aug. 18, 2011. All publications and patents mentioned herein are hereby incorporated by reference in their entirety as if each individual publication or patent was specifically and individually indicated to be incorporated by reference.

### FIELD OF THE INVENTION

Embodiments of the invention relate generally to an improved method for recovering organics from an aqueous phase. More particularly, embodiments of the invention relate to a method including one or more removal zones/stages employing sorbents or extractants for removal of organics from such aqueous phase and to methods of recovering such organics after removal. Embodiments of the invention also relate to methods for converting the lighter portion of the recovered organics to heavier organics.

### BACKGROUND OF THE INVENTION

With the rising costs and environmental concerns associated with fossil fuels, renewable energy sources have become increasingly important. The development of renewable fuel sources provides a means for reducing the dependence on fossil fuels. Accordingly, many different areas of renewable fuel research are currently being explored and developed.

With its low cost and wide availability, biomass has increasingly been emphasized as an ideal feedstock in renewable fuel research. Consequently, many different conversion processes have been developed that use biomass as a feedstock to produce useful biofuels and/or specialty chemicals. Existing biomass conversion processes include, for example, combustion, gasification, slow pyrolysis, fast pyrolysis, thermocatalytic pyrolysis, liquefaction, and enzymatic conversion. One of the useful products that may be derived from the aforementioned biomass conversion processes is a liquid product commonly referred to as “bio-oil.” Bio-oil may be processed into transportation fuels, hydrocarbon chemicals, and/or specialty chemicals.

In the conversion of biomass to bio-oils, even after the separation of the reaction products into bio-oil and aqueous phases, significant amounts of water-soluble organic compounds can be present in the aqueous phase. The loss of these organic compounds to the aqueous phase results in a decrease in the overall yield of bio-oil.

Accordingly, there is a need for an improved method for recovering organics from an aqueous stream produced in the conversion of biomass to a bio-oil.

In addition, the water-soluble organic compounds contain heavy oxygenated compounds and light oxygenated compounds having 5 or less carbon atoms. Such light oxygenated compounds having 5 or less carbon atoms only possess green chemicals value, but no value at all for fuels production (fuel value). In fact, trying to subject these light oxygenated compounds to hydrodeoxygenation for fuels production wastes hydrotreater capacity and valuable hydrogen, since only light gases can be produced. Similarly, reactions such as ketonization, etherification or esterification of these light oxygenated compounds will also render products of no fuel value.

Thus, there is also a need for an improved method for upgrading light oxygenated compounds recovered from an aqueous stream produced in the conversion of biomass to a bio-oil.

### BRIEF SUMMARY OF THE INVENTION

In accordance with an embodiment of the present invention, a method for recovering a water-soluble complex mixture of organic compounds from an aqueous stream is provided including:
a) passing the aqueous stream comprising a water-soluble complex mixture of organic compounds to a removal zone A for contact with a sorbent A comprising a polymeric microreticular sorbent resin for removal of at least a portion of the water-soluble complex mixture of organic compounds from the aqueous stream forming a removed quantity A comprising water-soluble organic compounds;
b) passing the aqueous stream from the removal zone A to a removal zone B for contact with a sorbent B for removal of at least a portion of the water-soluble complex mixture of organic compounds from the aqueous stream forming a removed quantity B comprising water-soluble organic compounds; and
c) recovering at least a portion of the removed quantity A from the removal zone A forming recovered quantity A and recovering at least a portion of the removed quantity B from the removal zone B forming recovered quantity B.

In accordance with another embodiment of the present invention, removed quantity B comprises light oxygenated compounds and heavy oxygenated compounds; wherein the light oxygenated compounds are separated from the removed quantity B in step c); and wherein a reactor feed comprises the light oxygenated compounds and is charged to a basic catalyzed reactor containing a basic catalyst for conversion of the light oxygenated compounds to heavier oxygenated compounds.

In accordance with another embodiment of the present invention, a method for recovering a water-soluble complex mixture of organic compounds from an aqueous stream is provided and includes:
a) separating a bio-oil/water stream comprising a water-soluble complex mixture of organic compounds, water-insoluble organic compounds, and water into a bio-oil stream comprising water-insoluble organic compounds and into the aqueous stream comprising a water-soluble complex mixture of organic compounds;
b) contacting the aqueous stream with an activated carbon-based sorbent for removal of at least a portion of the water-soluble complex mixture of organic compounds from the aqueous stream forming a removed quantity comprising water-soluble organic compounds, wherein the activated carbon-based sorbent has been surface treated in a manner resulting in a reduction in the number of polar and/or charged groups on the surface, and wherein at least 40% of the pore volume of the activated carbon-based sorbent results from pores having diameters in the range of from about 15 Å to about 50 Å; and
c) recovering at least a portion of the removed quantity from the activated carbon-based sorbent forming a recovered quantity; wherein the recovered quantity is combined with the bio-oil stream.

In accordance with another embodiment of the present invention, the recovered quantity comprises light oxygenated compounds and heavy oxygenated compounds; and wherein a reactor feed comprises the recovered quantity and is charged to a basic catalyzed reactor containing a basic catalyst for conversion of the light oxygenated compounds to heavier oxygenated compounds prior to combination with the bio-oil stream.

### BRIEF DESCRIPTION OF THE DRAWINGS

The features and advantages of embodiments of the invention will be illustrated with reference to the following drawings. The drawings are not to scale and certain features are shown exaggerated in scale or in schematic form in the interest of clarity and conciseness.

FIG. 1 is a schematic view of a two-stage sorption method/system for carrying out a specific embodiment of the invention.

FIG. 1a is a schematic view of a two-stage sorption method/system including up to two additional optional stages for carrying out specific embodiments of the invention.

FIG. 1b is a schematic view of a two-stage sorption method/system including up to two additional optional stages for carrying out specific embodiments of the invention.

FIG. 1c is a schematic view of a two-stage sorption method/system including up to two additional optional stages for carrying out specific embodiments of the invention.

FIG. 2 is a schematic view of a sorption method/system including a bio-oil/water separator for carrying out specific embodiments of the invention.

FIG. 3 is a schematic view of a method/system for recovering sorbed quantities from a removal zone in accordance with an embodiment of the invention.

FIG. 4 is a schematic view of a method/system for recovering sorbed quantities from a removal zone in accordance with an embodiment of the invention.

FIG. 5 is a schematic view of a method/system for recovering sorbed quantities from a removal zone in accordance with an embodiment of the invention.

FIG. 6 is a graph depicting thermograms for various granulated activated carbon samples.

FIG. 7 is a schematic view of a method/system for reacting recovered quantities from a removal zone(s) in accordance with an embodiment of the invention.

FIG. 8 is a schematic view of a method/system for reacting recovered quantities from a removal zone(s), which includes a separator, in accordance with an embodiment of the invention.

FIG. 9 is a schematic view of a method/system for reacting recovered quantities from a removal zone(s), which includes a separator, in accordance with an embodiment of the invention.

FIG. 10 is a schematic view of a method/system for removing, recovering and reacting oxygenated compounds contained in an aqueous stream in accordance with an embodiment of the invention.

FIG. 11 is a graph depicting Nuclear Magnetic Resonance spectra of a light oxygenated compound feed and the reaction product resulting from reacting such feed with a basic catalyst.

FIG. 12 presents a visual comparison of samples of the light oxygenated compound feed and the resulting reaction product of FIG. 11.

### DETAILED DESCRIPTION OF THE INVENTION

Of the various processes for converting biomass, pyrolysis processes, in particular flash pyrolysis processes, are generally recognized as offering the most promising routes to the conversion of solid biomass materials to liquid products, generally referred to as bio-oil or bio-crude. In addition to liquid reaction products, these processes produce gaseous reaction products and solid reaction products. Gaseous reaction products comprise carbon dioxide, carbon monoxide, and relatively minor amounts of hydrogen, methane, and ethylene. The solid reaction products comprise coke and char.

In order to maximize the liquid yield, while minimizing the solid and non-condensable gaseous reaction products, the pyrolysis process should provide a relatively fast heating rate of the biomass feedstock. Lately, the focus has been on ablative reactors, cyclone reactors, and fluidized reactors to provide the fast heating rates. Fluidized reactors include both fluidized stationary bed reactors and transport reactors.

Transport reactors provide heat to the reactor feed by injecting hot particulate heat carrier material into the reaction zone. This technique provides rapid heating of the feedstock. The fluidization of the feedstock ensures an even heat distribution within the mixing zone of the reactor.

The biomass to be pyrolyzed is generally ground to a small particle size in order to optimize pyrolysis. The biomass may be ground in a grinder or a mill until the desired particle size is achieved.

Regardless of the process used to produce bio-oil from the conversion of biomass, an aqueous stream is produced as a part of the reaction products. In the case of non-catalytic pyrolysis, this aqueous stream is usually emulsified with the organic portion (bio-oil) of the reaction products. In such case, the aqueous stream is only separable from the organic portion of the reaction products upon breaking the emulsion by some further treatment, such as hydrotreatment or de-oxygenation, of the organic components of the reaction products. In the case of catalytic pyrolysis of biomass, the aqueous and bio-oil streams form into two separate phases which are separable by methods including, but not limited to, gravity separation and centrifugation. In such case, the bio-oil phase is often more dense than the aqueous phase, causing the aqueous phase to rest on top of the bio-oil phase. In order to invert the layers (resulting in lower solids content for the bio-oil), the density of either the aqueous phase or bio-oil phase can be adjusted. Methods to perform such reversal of layers have been disclosed in U.S. patent application Ser. No. 13/212,861 filed on Aug. 18, 2011, which has been incorporated herein by reference in its entirety.

The aqueous stream, however obtained from the conversion of biomass, can contain up to 10 or up to 15 wt % of a water-soluble complex mixture of organic compounds. This amount of organic compounds can account for up to 30 wt % of the total organics yield from the biomass. The water-soluble complex mixture of organic compounds contained in the aqueous phase include phenols, catechols, aromatics, aldehydes, ketones, carboxylic acids, furans, indenols, and naphthols. Their relative proportions increase with increasing polarity of the compound.

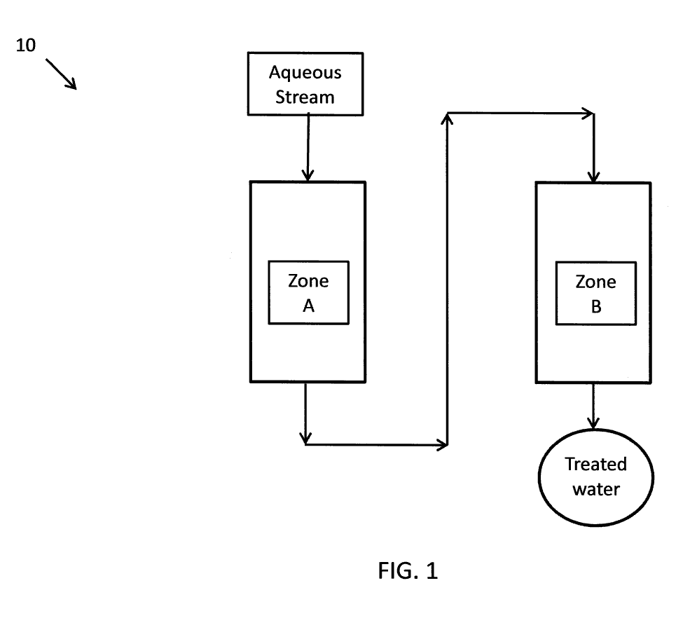

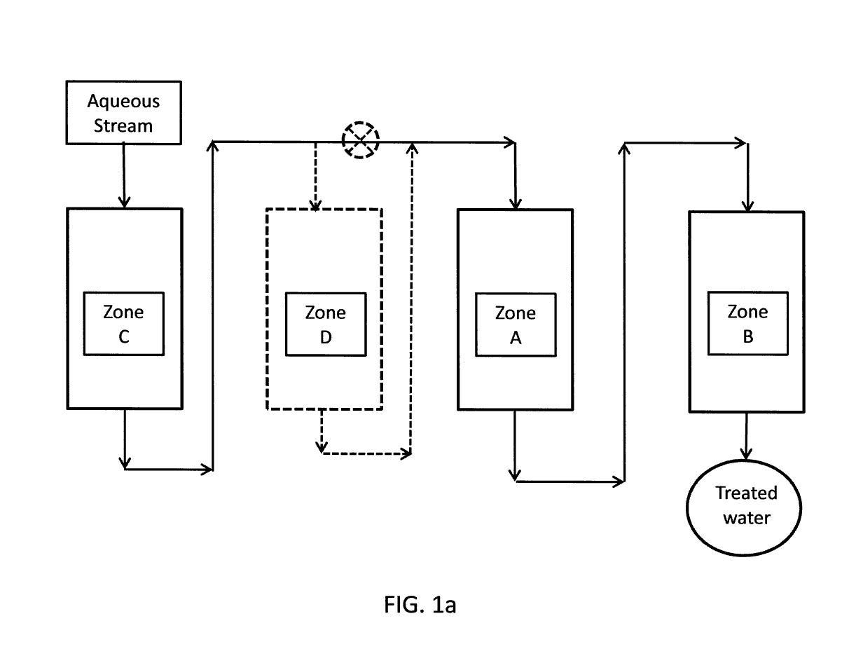

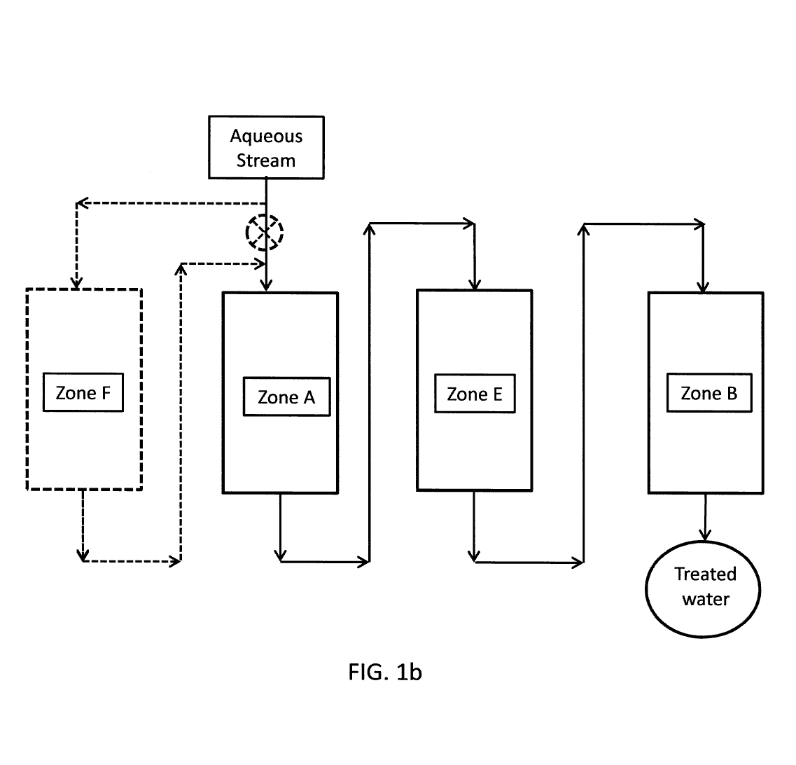

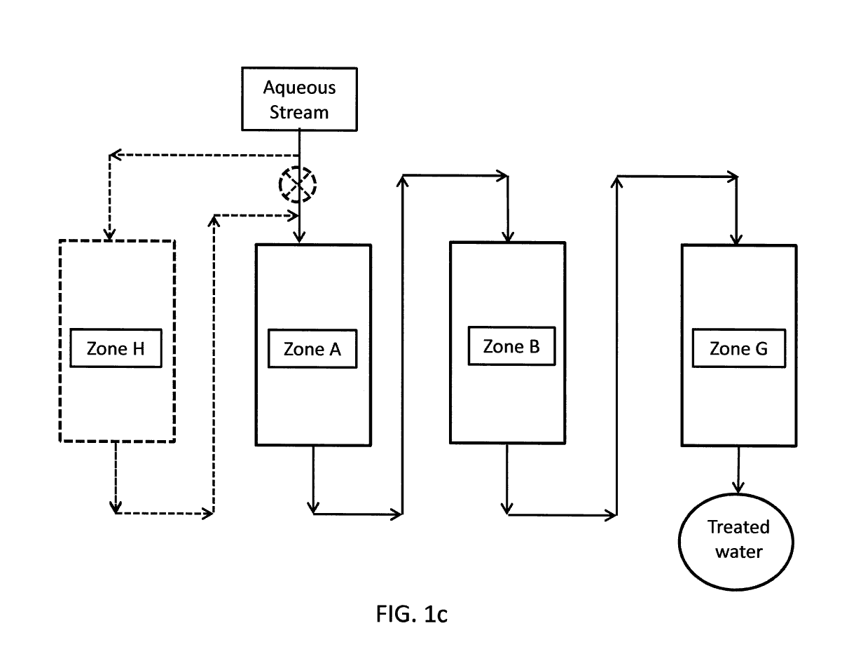

An embodiment of the invention will be described with reference to FIG. 1. A method 10 for recovering at least a portion of the water-soluble complex mixture of organic compounds from the aqueous stream comprises, consists of, or consists essentially of:
a) passing the aqueous stream comprising the water-soluble complex mixture of organic compounds to a removal zone A for contact with a sorbent A comprising a polymeric microreticular sorbent resin contained therein for removal of at least a portion of the water-soluble complex mixture of organic compounds from the aqueous stream forming a removed quantity A comprising water-soluble organic compounds; and
b) passing the aqueous stream from the removal zone A to a removal zone B for contact with a sorbent B contained therein for removal of at least a portion of the water-soluble complex mixture of organic compounds from the aqueous stream forming a removed quantity B comprising water-soluble organic compounds and a treated water stream.

At least a portion of the removed quantity A can be recovered from the removal zone A forming a recovered quantity A and at least a portion of the removed quantity B can be recovered from the removal zone B forming a recovered quantity B.

Sorbent B can be selected from the group consisting of polymeric microreticular sorbent resins, zeolite-based adsorbents, clay-based adsorbents, activated carbon-based sorbents, and mixtures thereof. The polymeric microreticular sorbent resins can be selected from the group consisting of Amberlite, Osorb, Amberlyst, Super adsorbent, and mixtures thereof; the zeolite-based adsorbents can be selected from the group consisting of X-Faujasite, Y-Faujasite, ZSM-5, zeolite-A, and mixtures thereof; the clay-based adsorbents can be selected from the group consisting of kaolin, bentonite, chlorite, perovskite, smectite, organoclays and mixtures thereof; and the activated carbon-based sorbents can be selected from the group consisting of microporous activated carbon, mesoporous activated carbon, carbon molecular sieves, carbon microbeads, carbon powder, granular activated carbon, and mixtures thereof.

With further reference to FIGS. 1 and 1a, the aqueous stream can optionally be passed to a removal zone C prior to being passed to removal zone A. In removal zone C, at least a portion of the water-soluble complex mixture of organic compounds can be removed from the aqueous stream forming a removed quantity C comprising water-soluble organic compounds. At least a portion of the removed quantity C can be removed from the removal zone C forming recovered quantity C.

Removal zone C can comprise a sorbent C selected from the group consisting of zeolite-based adsorbents (as described above), clay-based adsorbents, (as described above), and mixtures thereof, which sorbs at least a portion of the water-soluble complex mixture of organic compounds from the aqueous stream through contact with sorbent C forming the removed quantity C.

With further reference to FIGS. 1 and 1a, the aqueous stream can optionally be passed from removal zone C to a removal zone D comprising a super absorbent polymer for removal of at least a portion of the water from the aqueous stream through contact with the super absorbent polymer and yielding a stream of recovered quantity D comprising concentrated water-soluble organic compounds. The stream of recovered quantity D can then be passed to removal zone A as at least part of the aqueous stream. Also, the super absorbent polymer in removal zone D can be regenerated by taking removal zone D offline and heating at a temperature between about 50° C. and about 90° C. under an inert gas flow having a GHSV of at least about 0.5 h⁻¹, thereby removing the water.

Removal zone C can comprise an extraction zone wherein the aqueous stream is contacted with a solvent selected from the group consisting of i) renewable gasoline, ii) toluene, iii) xylene, iv) oxygenated solvents selected from the group consisting of methanol, ethanol, isopropanol, acetone, methylbutyl ketone, tetrahydrofuran, ethyl acetate, and v) mixtures thereof for extractive removal of at least a portion of the water-soluble complex mixture of organic compounds from the aqueous stream forming the removed quantity C.

With reference to FIGS. 1 and 1b, the aqueous stream can optionally be passed from removal zone A to a removal zone E comprising a super absorbent polymer prior to being passed to removal zone B. At least a portion of the water from the aqueous stream can be removed in removal zone E through contact with the super absorbent polymer and yielding a stream of recovered quantity E comprising concentrated water-soluble organic compounds. The stream of recovered quantity E can then be passed from removal zone E to removal zone B as at least part of the aqueous stream. Also, the super absorbent polymer in removal zone E can be regenerated by taking removal zone E offline and heating at a temperature between about 50° C. and about 90° C. under an inert gas flow having a GHSV of at least about 0.5 h⁻¹, thereby removing the water.

With further reference to FIGS. 1 and 1b, the aqueous stream can optionally be passed to a removal zone F comprising a zeolite-based adsorbent prior to being passed to removal zone A. At least a portion of the water-soluble complex mixture of organic compounds are sorbed from the aqueous stream through contact with the zeolite-based adsorbent forming a removed quantity F comprising water-soluble organic compounds. At least a portion of the removed quantity F can be removed from the removal zone F forming recovered quantity F.

With reference to FIGS. 1 and 1c, the aqueous stream can optionally be passed from removal zone B to a removal zone G comprising a super absorbent polymer. At least a portion of the water from the aqueous stream can be removed in removal zone G through contact with the super absorbent polymer and yielding a stream of recovered quantity G comprising concentrated water-soluble organic compounds. Also, the super absorbent polymer in removal zone G can be regenerated by taking removal zone G offline and heating at a temperature between about 50° C. and about 90° C. under an inert gas flow having a GHSV of at least about 0.5 h⁻¹, thereby removing the water.

With further reference to FIGS. 1 and 1c, the aqueous stream can optionally be passed to a removal zone H comprising a sorbent H prior to being passed to removal zone A. Sorbent H can be selected from the group consisting of zeolite-based adsorbents, clay-based adsorbents, and mixtures thereof. At least a portion of the water-soluble organic compounds from the aqueous stream can be sorbed in removal zone H through contact with the sorbent H forming a removed quantity H comprising water-soluble organic compounds. At least a portion of the removed quantity H can be removed from the removal zone H forming recovered quantity H.

Optionally, a bio-oil/water stream comprising a water-soluble complex mixture of organic compounds, water-insoluble organic compounds, and water is separated into a bio-oil stream comprising water-insoluble organic compounds and into the aqueous stream. The recovered quantities (recovered from the aqueous stream) described above can be combined with the bio-oil stream.

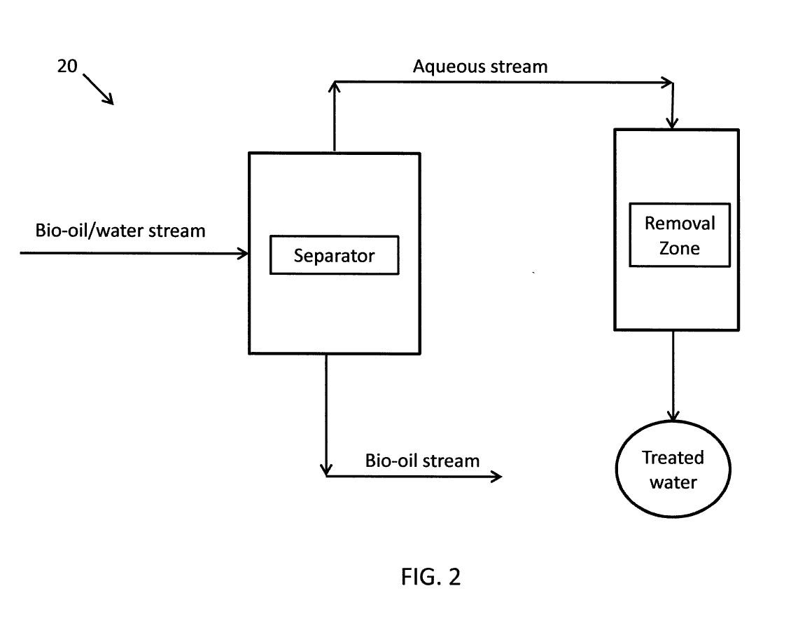

An embodiment of the invention will be described with reference to FIG. 2. A method 20 for recovering a water-soluble complex mixture of organic compounds from an aqueous stream comprises, consists of, or consists essentially of:
a) separating a bio-oil/water stream comprising a water-soluble complex mixture of organic compounds, water-insoluble organic compounds, and water in a separator into a bio-oil stream comprising water-insoluble organic compounds and into the aqueous stream comprising a water-soluble complex mixture of organic compounds;
b) contacting the aqueous stream with an activated carbon-based sorbent in a removal zone for removal of at least a portion of the water-soluble complex mixture of organic compounds from the aqueous stream forming a removed quantity comprising water-soluble organic compounds and a treated water stream, wherein the activated carbon-based sorbent has been surface treated in a manner resulting in a reduction in the number of polar and/or charged groups on the surface, and wherein at least about 40% of the pore volume of the activated carbon-based sorbent results from pores having diameters in the range of from about 15 Å to about 50 Å.

At least a portion of the removed quantity can be recovered from the activated carbon-based sorbent forming a recovered quantity, which can be combined with the bio-oil stream.

The aqueous stream, and intermediate streams described above, can be charged to the removal zones in either an upflow or a downflow mode.

With reference to FIGS. 1, 1a, 1b, 1c, and 2, the method of recovering the removed quantities from the sorbent materials can be any of the methods well known in the art, such as thermal desorption, thermal expansion (under vacuum) or chemical displacement.

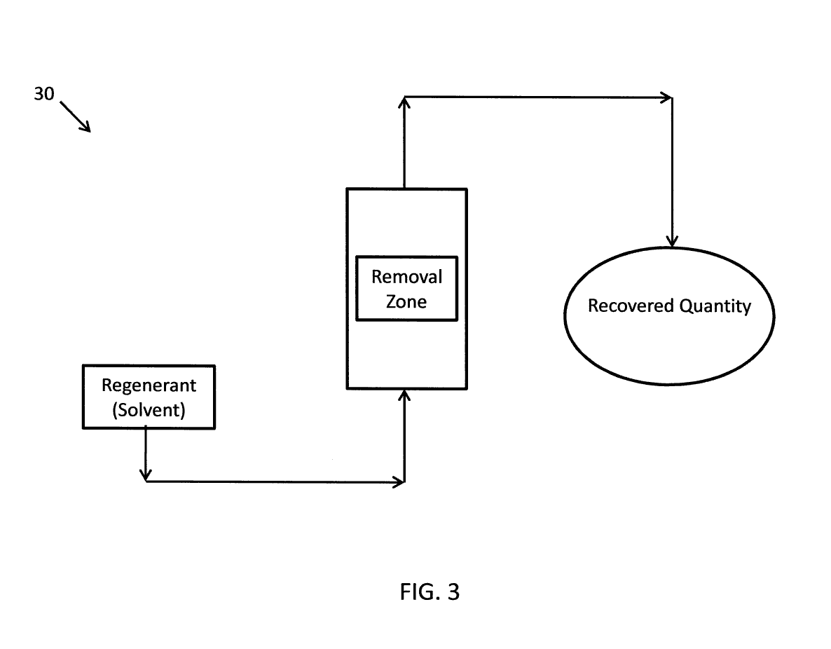

In accordance with an embodiment of the invention, a method 30 for recovering removed quantities from a removal zone is described below with reference to FIG. 3. When the sorbent in a removal zone 300 is selected from the group consisting of at least one of the polymeric microreticular sorbent resins, at least one of the activated carbon-based sorbents, and mixtures thereof, the recovery of the removed quantities as variously described above is carried out by chemical displacement at temperatures in the range of about 20° C. to about 200° C. or about 30° C. to about 150° C. or about 40° C. to about 100° C. using a regenerant (solvent) selected from the group consisting of i) renewable gasoline, ii) toluene, iii) xylene, iv) an oxygenated solvent selected from the group consisting of methanol, ethanol, isopropanol, acetone, methylbutyl ketone, methylisobutyl ketone, tetrahydrofuran, ethyl acetate, and v) mixtures thereof. The regenerant is charged to the removal zone displacing the removed quantity from the sorbent, and the removed quantity is recovered forming a recovered quantity. The regenerant can then be displaced from the sorbent by any suitable manner in order to prepare the removal zone for further removal of water-soluble organic compounds from the aqueous stream, once put back on-line.

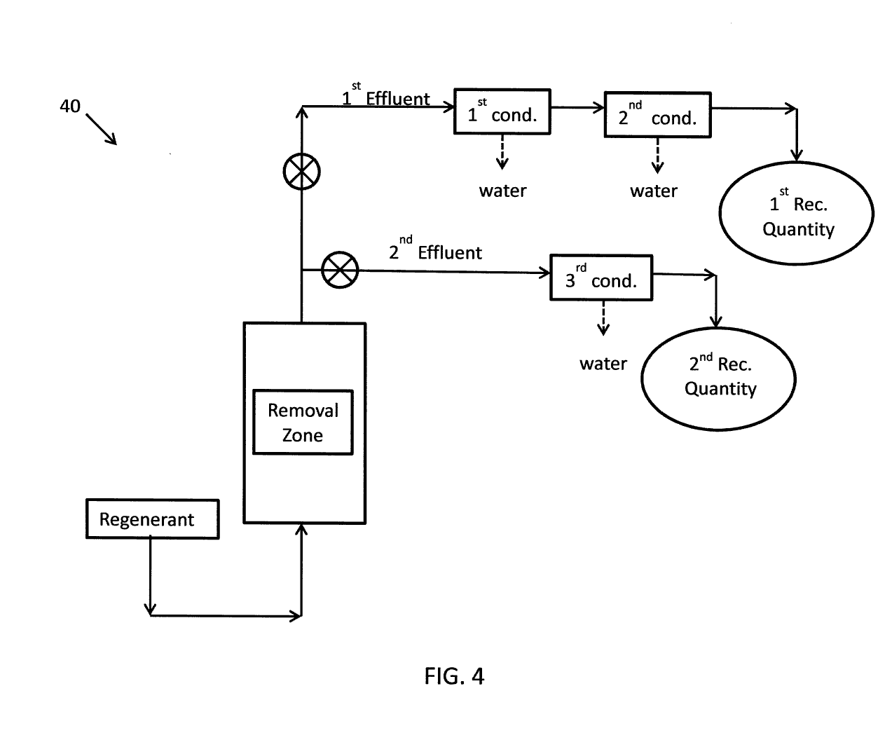

In accordance with an embodiment of the invention, a method 40 for recovering removed quantities from a removal zone is described below with reference to FIG. 4. When the sorbent in the removal zone is selected from the group consisting of at least one of the zeolite-based adsorbents, at least one of the clay-based adsorbents, at least one of the activated carbon-based sorbents, and mixtures thereof, the recovery of the removed quantities as variously described above is carried out by thermal desorption in accordance with the following.

i) The sorbent in the removal zone is heated to a temperature in the range of from about 20° C. to about 200° C. or about 50° C. to about 150° C. or about 60° C. to about 130° C., by introduction of a heated regenerant, which can be an inert gas, to the removal zone at pressures slightly above atmospheric pressure. A first effluent is removed from the removal zone and is partially condensed in a first condenser at a temperature in the range of from about 20° C. to about 50° C., for a period of time between about 0.2 to about 6 hours or about 0.5 to about 4 hours, followed by passing the first effluent to a second condenser wherein the first effluent is further partially condensed at a temperature in the range of from about −150° C. to about −30° C. or about −100° C. to about −40° C. for a period of time between about 0.2 to about 6 hours or about 0.5 to about 4 hours, forming a first recovered quantity.

ii) Thereafter, the sorbent is heated to a temperature in the range of from about 130° C. to about 500° C. or about 150° C. to about 400° C. or about 200° C. to about 350° C., under at least a partial vacuum and for a period of time between about 0.2 to about 6 hours or about 0.5 to about 4 hours. The vacuum can be up to about 0.01 or up to about 0.1 or up to about 1 torr. A second effluent is removed from the removal zone and is passed to a third condenser wherein the second effluent is partially condensed at a temperature in the range of from about −150° C. to about −30° C. or about −100° C. to about −40° C., forming a second recovered quantity. Condensed water can be drawn off from each of the first, second, and third condensers.

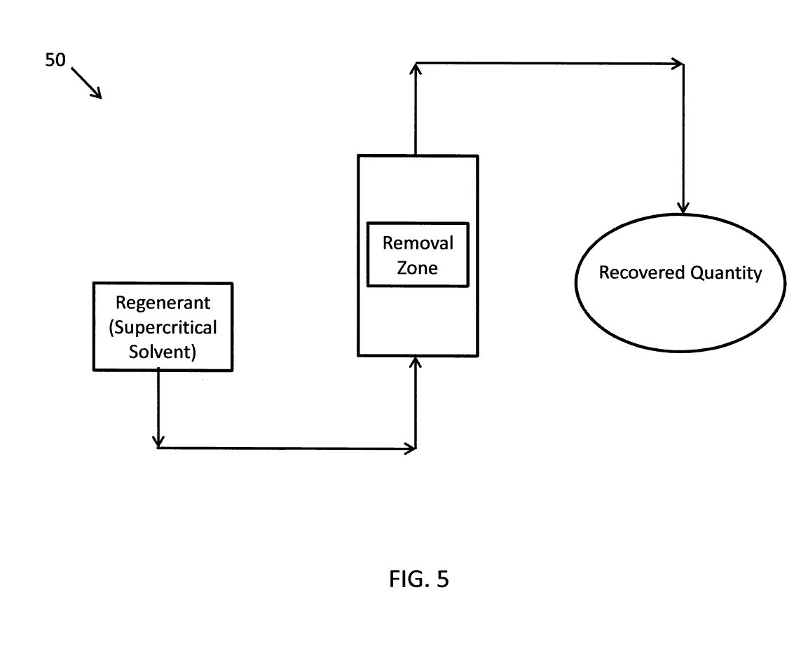

In accordance with an embodiment of the invention, a method 50 for recovering removed quantities from a removal zone is described below with reference to FIG. 5. When the sorbent in the removal zone is selected from the group consisting of at least one of the activated carbon-based sorbents, the recovery of the removed quantity is carried out by chemical displacement using a regenerant which is a supercritical solvent selected from the group consisting of supercritical CO₂, supercritical propane, supercritical butane, supercritical toluene, supercritical xylene, and mixtures thereof. The regenerant is charged to the removal zone displacing the removed quantity from the sorbent, and the removed quantity is recovered forming a recovered quantity. The regenerant can then be displaced from the sorbent by any suitable manner in order to prepare the removal zone for further removal of water-soluble organic compounds from the aqueous stream, once put back on-line.

The regenerant streams described above can be charged to the removal zones for recovery of the recovered quantities in either an upflow or a downflow mode.

Each of the recovered quantities described above can comprise, consist of, or consist essentially of light oxygenated compounds and heavy oxygenated compounds. The light oxygenated compounds can comprise, consist of, or consist essentially of compounds selected from the group consisting of ketones, aldehydes and carboxylic acids, but can also include other light oxygenated compounds. Typically, such light oxygenated compounds contain 5 or less carbon atoms, or between 2 and 5 carbon atoms. The heavy oxygenated compounds can comprise, consist of, or consist essentially of phenols, methoxy-substituted aromatics, anhydrosugars, benzofurans, and diols.

The polymeric microreticular sorbent resins described above, with the exception of the Superadsorbent resin, are more selective for the removal of the light oxygenated compounds than for the heavy oxygenated compounds from the aqueous stream. More particularly, the polymeric microreticular sorbent resins remove 50%, or 30%, or 20% more, by weight, of the light oxygenated compounds as compared to the heavy oxygenated compounds from the aqueous stream. The Superadsorbent resin is highly selective towards water.

The zeolite-based adsorbents described above are more selective for the removal of the heavy oxygenated compounds than for the light oxygenated compounds from the aqueous stream. More particularly, the zeolite-based adsorbents remove 50%, or 30%, or 20% more, by weight, of the heavy oxygenated compounds as compared to the light oxygenated compounds from the aqueous stream.

The clay-based adsorbents and the activated carbon-based sorbents remove both light and heavy oxygenated compounds, and do not selectively remove one over the other.

In order to convert such light oxygenated compounds to fuel-range compounds, only reactions that build up C—C bonds, which lead to at least the duplication of the skeleton length could be deemed valuable for fuels production. Condensations, additions and reductions are examples of such reactions.

During separation of the aqueous phase from the bio-oil phase, partition of the oxygenated compounds into the aqueous phase generally decreases with increasing molecular weight. Thus, the molar concentration of light oxygenated compounds present in the aqueous phase is greater than that of the heavy oxygenated compounds. The recovery of these oxygenated compounds by physical methods has been described above and all embodiments of this application can be used to yield a stream highly enriched in the light oxygenated compounds (60-98% oxygenates/2-40% water, or 70-96% oxygenates/4-30% water, or 80-95% oxygenates/5-20% water).

Reactions involving C—C bond formation of oxygenated compounds can occur in the presence of a basic catalyst. A reactor feed can comprise, consist of, or consist essentially of a component selected from the group consisting of any of the recovered quantities A-H described herein (and the “recovered quantity” from any of the other embodiments), or separated light oxygenate compound portions thereof, and combinations thereof. The reactor feed can comprise light oxygenated compounds and can be charged to a basic catalyzed reactor containing a basic catalyst for conversion of the light oxygenated compounds to heavier oxygenated compounds.

The basic catalyst can comprise, consist of, or consist essentially of a material selected from the group consisting of: the oxides, mixed oxides, hydroxides and mixed hydroxides of alkaline metals, alkaline earth metals, Group IIB metals, and Group IIIB metals; mixed oxides between Group IIIA or Group IVA metals with at least one element selected from the group consisting of alkaline metals, alkaline earth metals, Group IIB metals, and Group IIIB metals; mixed hydroxides between Group IIIA or Group IVA metals with at least one element selected from the group consisting of alkaline metals, alkaline earth metals, Group IIB metals, and Group IIIB metals; and mixtures thereof.

The basic catalyst can be a solid basic catalyst or a liquid basic catalyst. The solid basic catalysts are used in a heterogeneous phase, while the liquid basic catalysts are used in a homogeneous liquid phase. The liquid basic catalysts comprise, consist of, or consist essentially of aqueous solutions of the basic catalysts, and mixtures thereof. Non-limiting examples of solid basic catalysts are Na₂O, K₂O, MgO, CaO, SrO, BaO, ZrO₂, TiO₂, CeO, mixed oxides thereof such as MgO—ZrO₂, CeO—ZrO₂, TiO₂—ZrO₂, MgO—TiO₂, the corresponding solid hydroxides, other mixed oxides such as MgO—Al₂O3, MgO—SiO₂, CaO—Al₂O₃, CaO—SiO₂ and mixtures thereof.

Process conditions vary with the type of catalyst and reactor. The heterogeneous phase reactions can be carried out in a fixed bed reactor at relatively mild conditions, such as temperatures less than about: 450° C., or 400° C., or 350° C.; pressures less than about: 20 atm, or 10 atm, or 5 atm; and liquid hourly space velocities (LHSV) less than about: 30 h⁻¹, or 20 h⁻¹, or 10 h⁻¹. The homogeneous liquid phase reactions can be carried out in a fixed bed reactor in countercurrent flow or in a batch reactor (continuous, semi-continuous or discontinuous), under similar conditions.

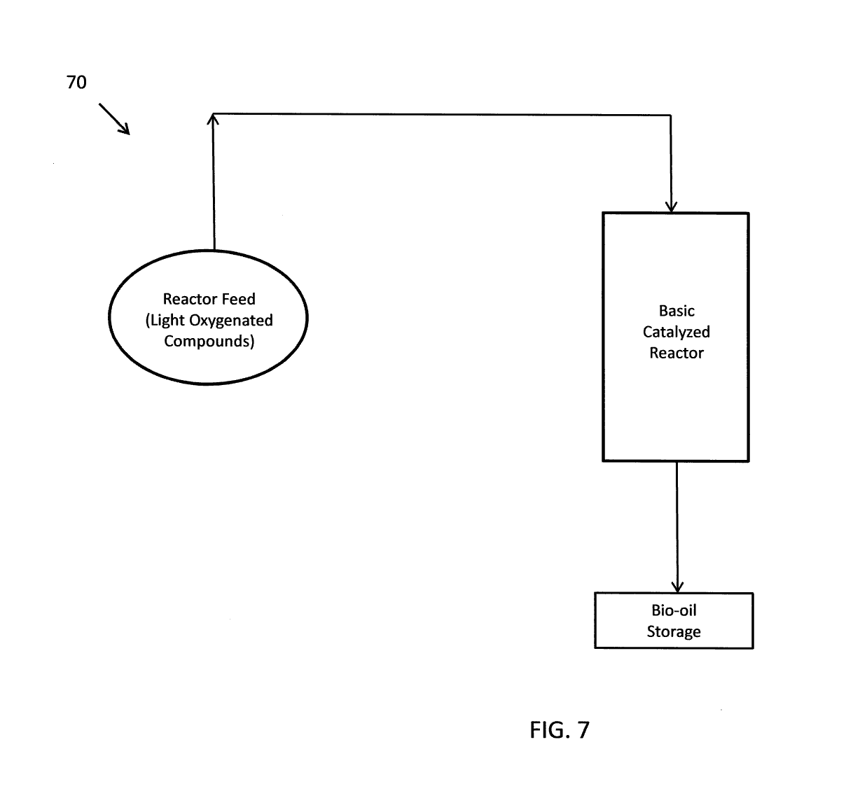

In accordance with an embodiment of the invention, a method 70 for upgrading the above described recovered quantities is described below with reference to FIG. 7. Any of the above described recovered quantities, or portions thereof, which comprise, consist of, or consist essentially of light oxygenated compounds can optionally be used alone or in any combination in this embodiment as the reactor feed which is charged to the Basic Catalyzed Reactor for contact with the basic catalyst described above. At least a portion of the light oxygenated compounds in the reactor feed are converted to heavier oxygenated compounds in the Basic Catalyzed Reactor, the effluent of which is charged to the Bio-oil Storage. The contents of the Bio-oil Storage vessel can then be deoxygenated to form fuel-range hydrocarbons.

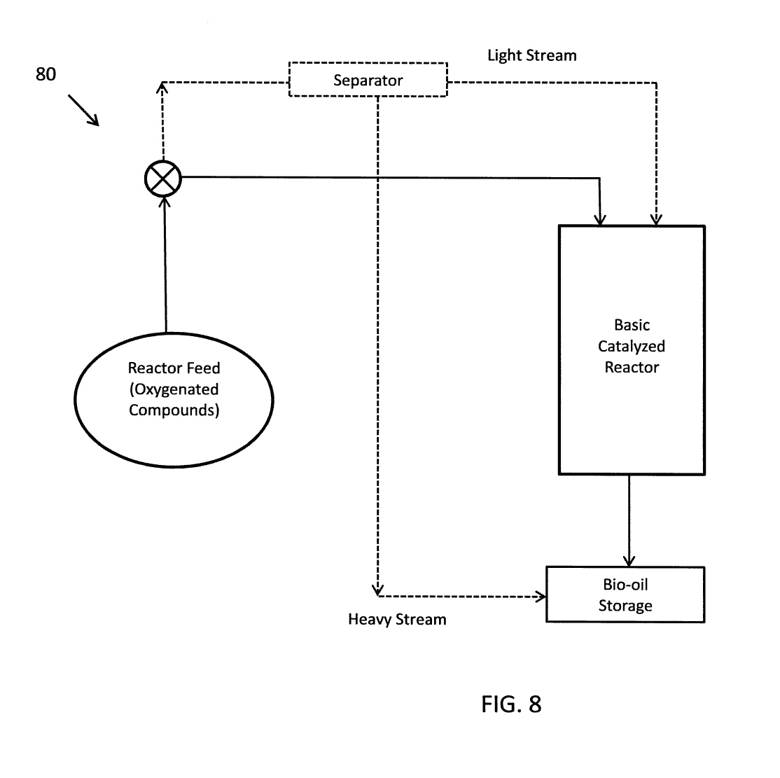

In accordance with an embodiment of the invention, a method 80 for upgrading the above described recovered quantities is described below with reference to FIG. 8. Any of the above described recovered quantities, or portions thereof, which comprise, consist of, or consist essentially of oxygenated compounds selected from the group consisting of light oxygenated compounds, heavy oxygenated compounds, and combinations thereof, can be used alone or in any combination in this embodiment as the reactor feed. Regarding the reactor feed, either: 1) at least a portion of the reactor feed is charged directly to the Basic Catalyzed Reactor for contact with the basic catalyst described above; or 2) at least a portion of the reactor feed is charged to a Separator for separation into a light stream comprising, consisting of, or consisting essentially of light oxygenated compounds and into a heavy stream comprising, consisting of, or consisting essentially of heavy oxygenated compounds; wherein the heavy stream is charged to the Bio-oil Storage and the light stream is charged to the Basic catalyzed reactor; or 3) both 1) and 2). At least a portion of the light oxygenated compounds are converted to heavier oxygenated compounds in the Basic Catalyzed Reactor, the effluent of which is charged to the Bio-oil Storage. The contents of the Bio-oil Storage vessel can then be deoxygenated to form fuel-range hydrocarbons.

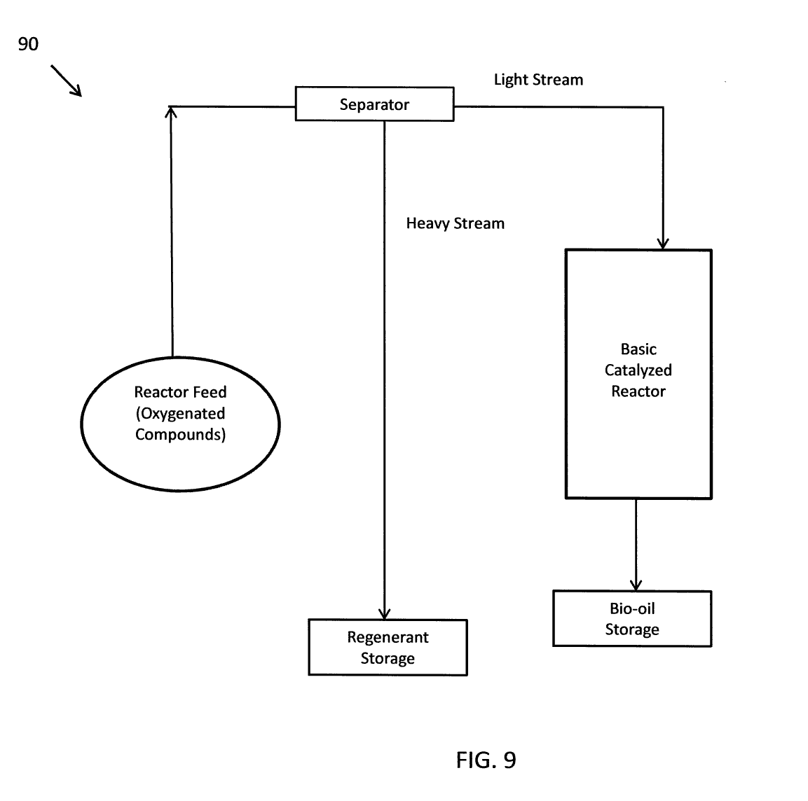

In accordance with another embodiment of the invention, a method 90 for upgrading a recovered quantity which has been recovered using chemical displacement and which comprises, consists of, or consists essentially of light oxygenated compounds and regenerant (chemical displacing solvent), is described below with reference to FIG. 9. Any such recovered quantity(ies) can be used as the reactor feed. At least a portion of the reactor feed is charged to a Separator for separation into a light stream comprising, consisting of, or consisting essentially of light oxygenated compounds and into a heavy stream comprising, consisting of, or consisting essentially of the regenerant (solvent); wherein the heavy stream is charged to the Regenerant Storage and the light stream is charged to the Basic Catalyzed Reactor. At least a portion of the light oxygenated compounds are converted to heavier oxygenated compounds in the Basic Catalyzed Reactor, the effluent of which is charged to the Bio-oil Storage. The contents of the Bio-oil Storage vessel can then be deoxygenated to form fuel-range hydrocarbons; and the regenerant can be recycled for use in recovering recovered quantities.

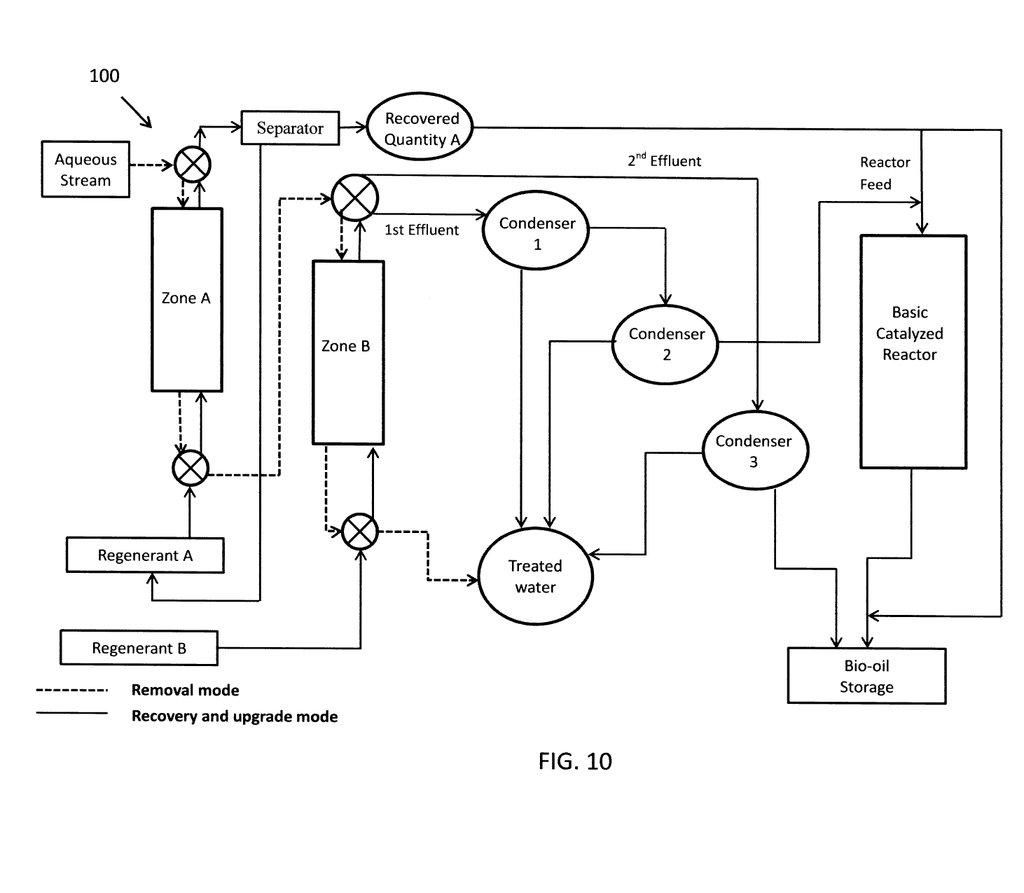

In accordance with an embodiment of the invention, a method 100 or removing at least a portion of the water-soluble complex mixture of organic compounds (comprising, consisting of, or consisting essentially of light oxygenated compounds and heavy oxygenated compounds) from the aqueous stream, and recovering and upgrading the removed quantities from removal zones A and B comprises, consists of, or consists essentially of the process described below with reference to FIG. 10.

Removal Mode:
a) passing the aqueous stream comprising, consisting of, or consisting essentially of the water-soluble complex mixture of organic compounds to removal zone A for contact with sorbent A, as described above, for removal of at least a portion of the water-soluble complex mixture of organic compounds from the aqueous stream forming a removed quantity A comprising heavy oxygenated compounds, and optionally light oxygenated compounds; and
b) passing the aqueous stream from the removal zone A to removal zone B for contact with sorbent B contained therein, as described above, for removal of at least a portion of the water-soluble complex mixture of organic compounds from the aqueous stream forming a treated water stream and a removed quantity B comprising light oxygenated compounds and heavy oxygenated compounds.

Recovery and Upgrade Mode:

At least a portion of removed quantity A contained in removal zone A is removed by chemical displacement in accordance with the process described in FIG. 3 by passing a Regenerant A selected from the group consisting of i) renewable gasoline, ii) toluene, iii) xylene, iv) an oxygenated solvent selected from the group consisting of methanol, ethanol, isopropanol, acetone, methylbutyl ketone, methylisobutyl ketone, tetrahydrofuran, ethyl acetate, and v) mixtures thereof, to removal zone A. The removed quantity A is then recovered using the Separator forming a recovered quantity A. Further, either: 1) the recovered quantity A becomes a part of a reactor feed which is charged to a Basic Catalyzed Reactor for conversion of any optionally present light oxygenate compounds, or 2) the recovered quantity A is charged to a Bio-oil Storage vessel, or 3) a portion of the recovered quantity A becomes a part of a reactor feed and is charged to a Basic Catalyzed Reactor for conversion of any optionally present light oxygenate compounds and a portion of the recovered quantity A is charged to a Bio-oil Storage vessel.

At least a portion of the removed quantity B contained in removal zone B can be removed by thermal desorption in accordance with the following:

i) removed quantity B in the removal zone B is heated to a temperature in the range of from about 20° C. to about 200° C. or about 50° C. to about 150° C. or about 60° C. to about 130° C., by introduction of a heated regenerant, which can be an inert gas, to the removal zone B at pressures slightly above atmospheric pressure. A first effluent is removed from the removal zone B and is partially condensed in a first condenser at a temperature in the range of from about 20° C. to about 50° C., for a period of time between about 0.2 to about 6 hours or about 0.5 to about 4 hours, followed by passing the first effluent to a second condenser wherein the first effluent is further partially condensed at a temperature in the range of from about −150° C. to about −30° C. or about −100° C. to about −40° C. for a period of time between about 0.2 to about 6 hours or about 0.5 to about 4 hours, forming a first recovered quantity. The first recovered quantity comprises light oxygenated compounds and optionally heavy oxygenated compounds. The first recovered quantity is then charged, as at least a part of the reactor feed, to the Basic Catalyzed Reactor. At least a portion of the light oxygenated compounds are converted to heavier oxygenated compounds in the Basic Catalyzed Reactor. The heavier oxygenated compounds from the Basic Catalyzed Reactor are charged to the Bio-oil Storage.

ii) thereafter, removed quantity B is heated to a temperature in the range of from about 130° C. to about 500° C. or about 150° C. to about 400° C. or about 200° C. to about 350° C., under at least a partial vacuum and for a period of time between about 0.2 to about 6 hours or about 0.5 to about 4 hours. The vacuum can be up to about 0.01 or up to about 0.1 or up to about 1 torr. A second effluent is removed from the removal zone B and is passed to a third condenser wherein the second effluent is partially condensed at a temperature in the range of from about −150° C. to about −30° C. or about −100° C. to about −40° C., forming a second recovered quantity comprising heavy oxygenated compounds, and optionally light organic compounds. The second recovered quantity is charged to the Bio-oil Storage. The contents of the Bio-oil Storage can then be deoxygenated to form fuel-range hydrocarbons. Also, condensed water can be drawn off from each of the first, second, and third condensers and combined with the treated water.

### EXAMPLES

### Example 1

Southern yellow pine wood particles were thermocatalytically converted to a reaction product, which was then separated into a bio-oil stream and into an aqueous stream containing about 10 wt % water-soluble organic compounds. A volume of 25 ml of the aqueous stream was then contacted with about 2.5 g of fresh granulated activated carbon (fresh GAC) resulting in a spent GAC containing the water-soluble organic compounds. A 5.06 g quantity of the spent GAC was filtered off from the aqueous phase and subjected to vacuum desorption in a vacuum oven under a vacuum of about 0.1 torr and at a temperature of 120° C., to form vacuum regenerated GACs. A similar experiment was carried out in parallel and the 5.01 g of filtered off spent GAC was subjected to vacuum desorption in such vacuum oven under a vacuum of about 0.1 torr and at a temperature of 50° C. The results of such vacuum desorption are shown in Table 1 below. For the 120° C. run, the change in weight loss % from 60 minutes to 120 minutes of only about 0.5% (100*(44.83-44.59)/44.59) shows that the recoverable material from the spent GAC was substantially completely recovered after two hours treatment. Table 1 also shows that at 30 minutes for the 120° C. run 98% of the recoverable material was desorbed. For the 50° C. run, Table 1 shows that after 180 minutes 93% of the recoverable material was desorbed. Thus, given enough time for desorption, this data shows that vacuum desorption at temperatures as low as 50° C. is effective in removing recoverable water-soluble organic compounds from a spent GAC.

TABLE 1

| | Conditions | | | |
|---|---|---|---|---|
| | 120° C. Vacuum Oven | | 50° C. Vacuum Oven | |
| Total Time | GAC Mass | | GAC Mass | Weight loss, |
| (min) | (g) | Weight loss, % | (g) | % |
| 0 | 5.06 | 0 | 5.01 | 0 |
| 30 | 2.84 | 43.87 | 4.04 | 19.38 |
| 60 | 2.80 | 44.59 | 3.29 | 34.41 |
| 120 | 2.79 | 44.83 | — | |
| 180 | — | | 2.93 | 41.52 |

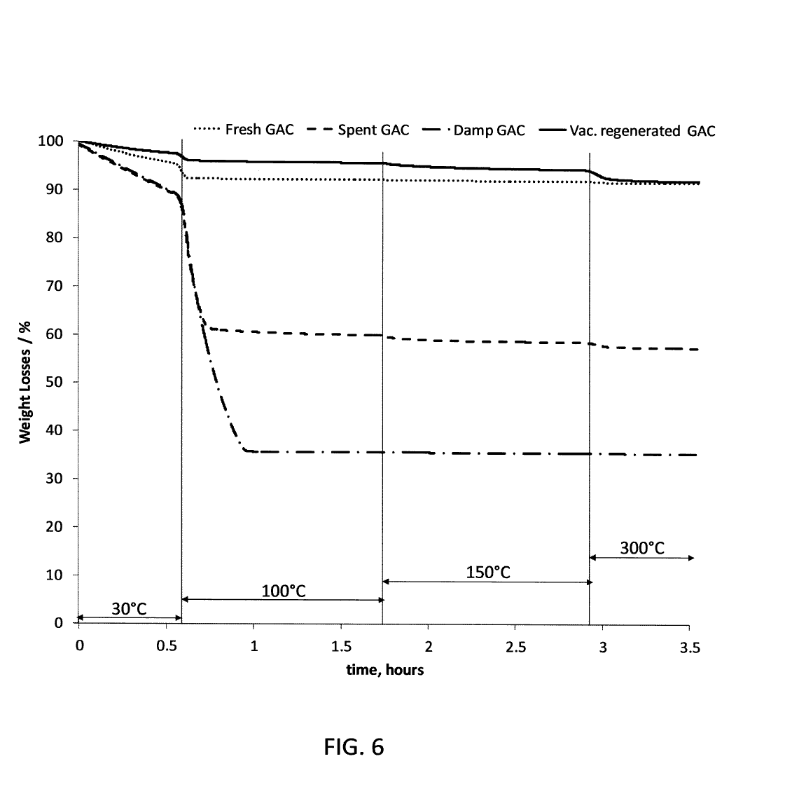

Thermogravimetric analysis (TGA) thermograms were obtained for samples of the spent GAC, the vacuum regenerated GAC from the 120° C. run, fresh GAC, and a fresh GAC made damp with DI water (damp GAC). The thermograms are shown in FIG. 6. As can be seen in FIG. 6, the weight loss for the spent GAC confirm the efficiency of thermal regeneration shown in Table 1, and that of the vacuum regenerated GAC was very low and similar to that of the fresh GAC, indicating vacuum regeneration is very effective at removing these water-soluble organic compounds from spent GAC.

### Example 2

A model aqueous solution was prepared containing 0.5 wt % phenol and about 1 wt % catechol. About 50 g quantity of this solution was charged at a flow rate of 1 ml/min to a vessel containing about 10 g of a mixture of sorbent resins referred to generally as Amberlite XAD (75% of the resin with product designation Amberlite XAD 761 and 25% of the resin with product designation Amberlite XAD 1600) which are manufactured by the Rohm and Haas company. The resulting spent sorbent resin mixture contained 0.16 g of phenol and 0.45 g of catechol. The spent sorbent resin mixture was subjected to desorption by extraction with about 8 ml ethanol. For both phenol and catechol, Table 2 below shows the wt % removal from the model solution, the wt % desorption from the sorbent resin mixture, and the total overall recovery. The data in Table 2 show that the Amberlite XAD resins are effective in removing phenol and catechol from an aqueous stream, and that such absorbed components can be effectively removed by chemical displacement with a suitable solvent from such resins.

TABLE 2

| Phenol | | | Catechol | | |
|---|---|---|---|---|---|
| | | Wt % | | Wt % | Wt % |
| Wt % | Wt % | Total | Wt % | De- | Total |
| Removal | Desorption | Recovery | Removal | sorption | Recovery |
| 94.8 | 48.5 | 46.0 | 71.4 | 60.6 | 43.3 |

For the following Examples 3, 4, 5 and 6, aqueous streams containing from about 7 to about 14 wt % water-soluble organic compounds were separated from reaction products resulting from the thermocatalytic conversion of southern yellow pine wood particles. The aqueous streams were then contacted with various sorbents for removal of water-soluble organic compounds, or in the case of Super Absorbent Polymer (SAP) the removal of water resulting in a concentrating effect of the water-soluble organic compounds.

### Example 3

Organics were removed from the aqueous stream following the scheme:

Feed→NaX zeolite→Osorb resin→Amberlyst resin

Table 3 below shows the selectivities for these sorbents in the removal of specific water-soluble organic compounds. The removal %'s for each sorbent is based on the amounts of the subject organics contained in the feed to that in the treated water. The data show that NaX zeolite is very effective in removing heavier compounds, Osorb resin is effective in removing phenolics, and that Amberlyst 21 resin is effective in removing acidic compounds.

TABLE 3

| | Removal, wt % | | |
|---|---|---|---|
| Compound Families | NaX Zeolite | Osorb resin | Amberlyst resin |
| Ketones and | 0 | 49 | 0 |
| Aldehydes | | | |
| Carboxylic acids | 56 | 38 | 86 |
| Phenolics | 71 | 84 | 100 |
| Unidentified heavy | 88 | 100 | N/A |
| compounds | | | |

### Example 4

Tables 4a, 4b, and 4c below show the total removal of water-soluble organic compounds from such aqueous streams described above resulting from the serial contact of the aqueous streams with different sequences and combinations of sorbents. As can be seen from Table 4a, the arrangement used in Run C demonstrated a very selective separation, and in consideration of the selectivities of the sorbents used (shown in Table 3), leaving most of the heavy compounds on the zeolite sorbent, most of the hydroxylic compounds on the Amberlyst sorbent and produced a concentrated ketones and aldehydes stream. The removal wt % for ketones and aldehydes in Run C is negative due to the manner in which the removal %'s are calculated, that is:

[(amount in the feed)−(amount in the effluent)]/(amount in the feed)

In the case of Run C, the SAP removes substantial amounts of water, thus concentrating the water-soluble organics not removed upstream by the zeolite. The hydroxylic compounds are then mostly removed by the following Amberlyst resin, leaving a concentrated stream of ketones and aldehydes having a concentration in the final effluent which is higher than the concentration in the feed due to the removal of water by the SAP. Similarly, comparing D and E data shown in Table 4b confirmed the advantageous effectiveness of using SAP in that last stage.

TABLE 4a

| | Removal, wt % | | |
|---|---|---|---|
| | RUN | | |
| | A | B | C |
| | Osorb resin/ | NaX Zeolite/ | NaX Zeolite/ |
| Compound | Amberlyst | Osorb resin/ | SAP/ |
| Families | resin | Amberlyst resin | Amberlyst resin |
| Ketones and | 80 | 16 | −38 |
| Aldehydes | | | |
| Carboxylic acids | 98 | 96 | 99 |
| Phenolics | 98 | 100 | 100 |
| Unidentified heavy | 92 | 100 | 100 |
| compounds | | | |

TABLE 4b

| | Removal, wt % | | |
|---|---|---|---|
| | RUN | | |
| | | | F |
| | D | E | NaX-Zeolite/ |
| | Osorb resin/ | Osorb resin/ | Osorb resin/ |
| | SAP/ | Amberlyst | SAP/Amberlyst |
| Compound Families | Amberlyst resin | resin/SAP | resin |
| Ketones and | 58 | 98 | 26 |
| Aldehydes | | | |
| Carboxylic acids | 97 | 98 | 100 |
| Phenolics | 100 | 92 | 100 |
| Unidentified heavy | 64 | 100 | 100 |
| compounds | | | |

### Example 5

Table 5 below shows the total removal and total recovery percentages of water-soluble organic compounds from an aqueous stream as described above resulting from the contact of the aqueous stream, in a column containing a commercial granulated activated carbon (GAC) manufactured by Norit Inc. and having product designation GAC300. The adsorber column was loaded in the downflow mode. Organic compounds were recovered from the GAC through thermal desorption carried out by:
1) establishing a nitrogen flow through the column and heating the column to ramped temperatures of about 90° C., then about 100° C., and then about 150° C., condensing and recovering organics from the effluent from the column in a first flask (condenser) at room temperature (about 21° C.) followed by further condensation and recovery of organics from the effluent in a second condenser at a temperature of about −78° C., and disconnecting the first and second condensers upon no further organics collection; and
2) heating the column to ramped temperatures of about 150° C. up to about 190° C. under nitrogen flow, and condensing and recovering organics from the effluent from the column in a third condenser connected to a vacuum pump and at a temperature of about −78° C.

The data in Table 5 shows that microporous GAC is effective in removing substantially all of the organics present and is selective for the recovery of the light organic compounds from an aqueous stream. The data also shows that such absorbed light components can be effectively removed from the GAC by thermal desorption.

TABLE 5

| | Removal, | Recovery, wt % | |
|---|---|---|---|
| Compound | wt % | | Grouped Totals |
| Lights | | | |
| Methyl Vinyl Ketone | 81 | 59 | 62 |
| 2-Butanone | 99 | 95 | (Total for lights) |
| Acetic Acid | 70 | 107 | |
| Benzene | 100 | 29 | |
| 1-Hydroxy-2-pentanone | 78 | 17 | |
| Propionic Acid | 97 | 64 | |
| Heavies | | | |
| Toluene | 79 | 21 | 5 |
| 2-Cyclopentan-1-one | 93 | 16 | (Total for heavies) |
| Phenol | 100 | 1 | |
| o-Cresol | 100 | 0 | |
| (p + m)-Cresol | 100 | 0 | |
| Catechol | 100 | 0 | |
| 1,4-Benzenediol | 100 | 0 | |
| 4-Methyl-1,2-benzediol | 100 | 0 | |
| Total for all organics | 93 | 29 | 33 |

### Example 6

Table 6 below shows the total removal and total recovery percentages of water-soluble organic compounds from an aqueous stream as described above resulting from the contact of the aqueous stream, in a column, with: 1) a mixture of sorbent resins referred to generally as Amberlite XAD (75% of the resin with product designation Amberlite XAD 761 and 25% of the resin with product designation Amberlite XAD 1600) which are manufactured by the Rohm and Haas company, followed by contact with: 2) a commercial granulated activated carbon (GAC) manufactured by Norit Inc. and having product designation GAC300. The bottom 25 vol % of the column was packed with the GAC and the top 75 vol % of the column was packed with the Amberlite XAD resin mixture. The adsorber column was loaded in the downflow mode. The spent adsorbents bed was subjected to desorption for recovery of organic compounds by extraction with ethanol in the upflow mode. Such recovery method was expected to be effective with regard to organics recovery from the Amberlite XAD sorbent, but have only minimal effectiveness in organics recovery from the GAC.

The data in Table 6 shows that the use of a two-stage system including Amberlite XAD resins and GAC is effective in removing and recovering organic compounds, including phenol and catechol, from an aqueous stream, and that such absorbed heavy components can be effectively removed from the Amberlite XAD by chemical displacement with a suitable solvent. The light compounds contained in the GAC can be recovered from the GAC by thermal desorption, as described in Examples 1 and 5.

TABLE 6

| | | Recovery, wt % | |
|---|---|---|---|
| Compound | Removal, wt % | | Grouped Totals |
| Lights | | | |
| Formaldehyde | 78.7 | 10.7 | 36.2 |
| Acetaldehyde | 95.3 | 22.1 | (Total for lights) |
| Methyl Vinyl Ketone | 90.9 | 50.4 | |
| 2-Butanone | 92.7 | 64.0 | |
| Acetic Acid | 92.7 | 15.0 | |
| Benzene | 98.4 | 73.6 | |
| 1-Hydroxy-2-pentanone | 92.0 | 26.9 | |
| Propionic Acid | 99.6 | 26.9 | |
| Heavies | | | |
| Toluene | 98.1 | 39.7 | 47.3 |
| 2-Cyclopentan-1-one | 99.7 | 30.0 | (Total for heavies) |
| Phenol | 100 | 39.2 | |
| o-Cresol | 100 | 60.7 | |
| (p + m)-Cresol | 100 | 55.5 | |
| Catechol | 100 | 34.6 | |
| 1,4-Benzenediol | 100 | 93.5 | |
| 4-Methyl-1,2-benzediol | 84.9 | 25.2 | |
| Total for all organics | 93.6 | 29.6 | |

### Example 7

A biomass feedstock of Southern Yellow Pine wood particles were thermocatalytically converted to a reaction product containing a bio-oil phase and an aqueous phase. The aqueous phase was obtained by gravity separation from the bio-oil phase. A stream of recovered light oxygenated compounds was obtained from treating the aqueous phase by first contacting with a mixture of Amberlite XAD resins followed by contact with GAC, in the same way as that described in Examples 5 and 6 above. The GAC was subjected to two-stage thermal desorption in accordance with Example 5, and the effluent resulting from the first desorption are the recovered light oxygenated compounds. The concentration of the recovered light oxygenated compounds from the first desorption is presented in Table 7.

TABLE 7

| | Recovered Light Oxygenated Compounds |
|---|---|
| | Composition (wt %) |
| Formaldehyde | 0.4 |
| Acetaldehyde | 22.5 |
| 2-butanone | 6.8 |
| Acetic acid | 3.3 |
| Propanoic acid | 1.3 |
| Water | 8.3 |

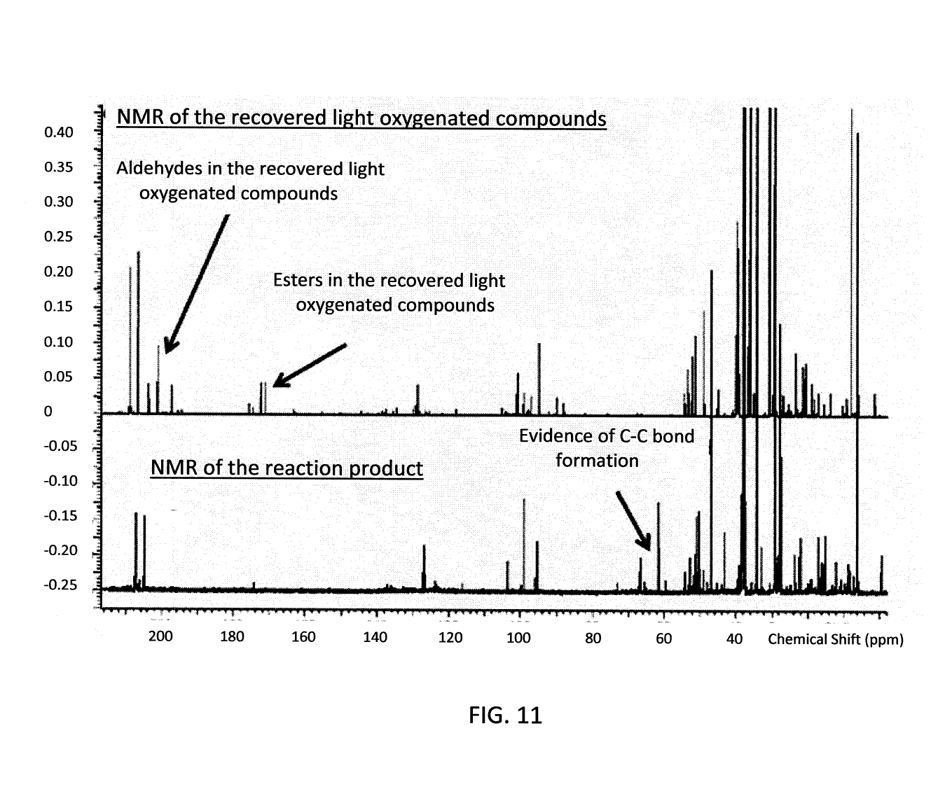

A 1 ml quantity of the recovered light oxygenated compounds was allowed to react, at 27° C. and 1 atm, in the presence of 80 mg of a solid NaOH pearl catalyst forming a reaction product. A sample of the recovered light oxygenated compounds feed and a sample of the reaction product were subjected to testing by Nuclear Magnetic Resonance (NMR). The H-NMR spectrum of the reaction product is compared to that of the recovered light oxygenated compounds feed in FIG. 11. NMR is commonly used to assess changes in composition resulting from treatment by various means. Functional groups of interest give signals in different regions of the NMR spectrum. Carbonyls typically appear between 170 and 210 in ¹³C NMR, while peaks in the 60-80 region are assigned to carbons singly bonded to oxygen.

As can be seen in FIG. 11, the conversion of carbonyl-bearing molecules is evidenced by the disappearance of the 170 and 210 peaks, which along with the introduction of new peaks in the 60-80 region suggests their transformation into alcohols (which include carbon singly bonded to oxygen rather than the double bonded carbon to oxygen bonds of the carbonyls). The absence of reducing conditions in the reactor along with the absence of volatile alcohols in the final reaction product strongly supports these new compounds being the product of C—C bond forming reactions between two smaller molecules, showing evidence of both the disappearance of aldehydes/ketones and associated C—C bond formation.

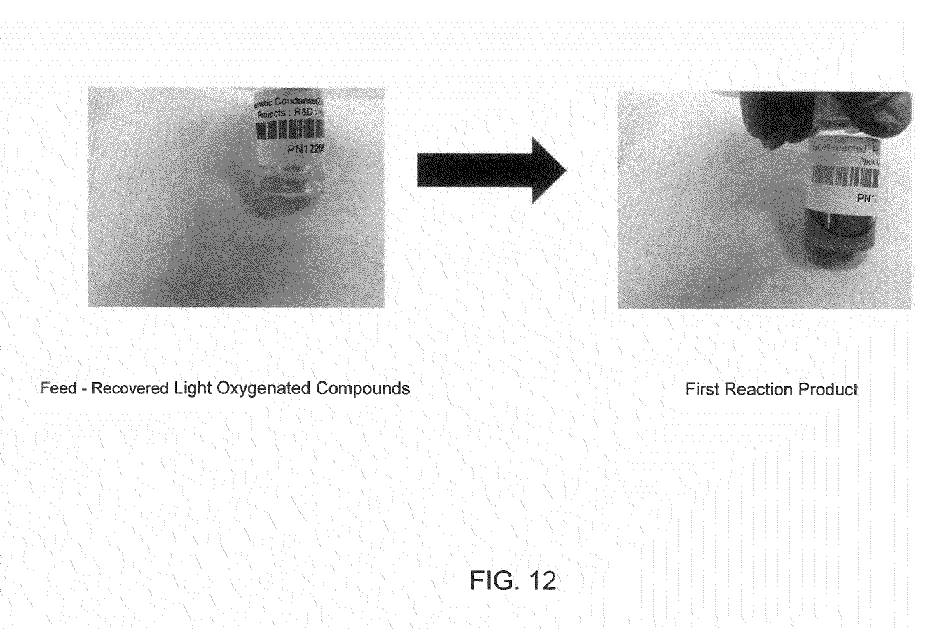

FIG. 12 presents a visual comparison of the recovered light oxygenated compounds feed and the reaction product. The recovered light oxygenated compounds feed was a mostly transparent homogeneous liquid, whereas the reaction product clearly shows an oil phase floating on top of an aqueous phase. This serves as confirmation that the homogeneous recovered light oxygenated compounds in the feed are converted, at least in part, to heavier oxygenated compounds which float on top of an aqueous phase.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

Further, unless expressly stated otherwise, the term “about” as used herein is intended to include and take into account variations due to manufacturing tolerances and/or variabilities in process control.

Changes may be made in the construction and the operation of the various components, elements and assemblies described herein, and changes may be made in the steps or sequence of steps of the methods described herein without departing from the spirit and the scope of the invention as defined in the following claims.

That which is claimed is:

1. A method comprising:
a) passing an aqueous stream comprising a water-soluble complex mixture of organic compounds to a removal zone A for contact with a sorbent A comprising a polymeric microreticular sorbent resin for removal of at least a portion of the water-soluble complex mixture of organic compounds from said aqueous stream forming a removed quantity A comprising water-soluble organic compounds;
b) passing said aqueous stream from said removal zone A to a removal zone B for contact with a sorbent B for removal of at least a portion of the water-soluble complex mixture of organic compounds from said aqueous stream forming a removed quantity B comprising water-soluble organic compounds;
c) recovering at least a portion of said removed quantity A from said removal zone A forming recovered quantity A and recovering at least a portion of said removed quantity B from said removal zone B forming recovered quantity B;
d) utilizing a reactor feed comprising a component selected from the group consisting of: at least a portion of said recovered quantity A, at least a portion of said recovered quantity B, and combinations thereof, wherein said reactor feed comprises light oxygenated compounds; and
e) charging said reactor feed to a basic catalyzed reactor containing a basic catalyst for conversion of said light oxygenated compounds to heavier oxygenated compounds.

2. The method of claim 1 wherein said basic catalyst comprises a material selected from the group consisting of: the oxides, mixed oxides, hydroxides and mixed hydroxides of alkaline metals, alkaline earth metals, Group IIB metals, and Group IIIB metals; mixed oxides between Group IIIA or Group IVA metals with at least one element selected from the group consisting of alkaline metals, alkaline earth metals, Group IIB metals, and Group IIIB metals; mixed hydroxides between Group IIIA or Group IVA metals with at least one element selected from the group consisting of alkaline metals, alkaline earth metals, Group IIB metals, and Group IIIB metals; and mixtures thereof.

3. The method of claim 1 wherein, prior to step a), said aqueous stream is passed to a removal zone C for removal of at least a portion of the water-soluble complex mixture of organic compounds from said aqueous stream forming a removed quantity C comprising water-soluble organic compounds; wherein step c) further comprises recovering at least a portion of said removed quantity C from said removal zone C forming recovered quantity C.

4. The method of claim 3 wherein said reactor feed optionally further comprises at least a portion of said recovered quantity C.

5. The method of claim 3 wherein, prior to step a), said aqueous stream is passed from removal zone C to a removal zone D comprising a super absorbent polymer for removal of at least a portion of the water from said aqueous stream through contact with said super absorbent polymer and yielding a stream of recovered quantity D comprising concentrated water-soluble organic compounds; and further comprising regenerating said super absorbent polymer by heating at a temperature between about 50° C. and about 90° C. under an inert gas flow having a GHSV of at least about 0.5 h⁻¹.

6. The method of claim 5 wherein said stream of recovered quantity D is utilized as said aqueous stream passed to said removal zone A in step a).

7. The method of claim 5 wherein said reactor feed optionally further comprises at least a portion of said recovered quantity D.

8. The method of claim 1 wherein, following step a) and prior to step b), said aqueous stream is passed to a removal zone E comprising a super absorbent polymer for removal of at least a portion of the water from said aqueous stream through contact with said super absorbent polymer and yielding a stream of recovered quantity E comprising concentrated water-soluble organic compounds; and further comprising regenerating said super absorbent polymer by heating at a temperature between about 50° C. and about 90° C. under an inert gas flow having a GHSV of at least about 0.5 h⁻¹.

9. The method of claim 8 wherein said stream of recovered quantity E is utilized as said aqueous stream passed to said removal zone B in step b.

10. The method of claim 8 wherein said reactor feed optionally further comprises at least a portion of said recovered quantity E.

11. The method of claim 8 wherein, prior to step a), said aqueous stream is passed to a removal zone F comprising a zeolite-based adsorbent which sorbs at least a portion of the water-soluble complex mixture of organic compounds from said aqueous stream through contact with said zeolite-based adsorbent forming a removed quantity F comprising water-soluble organic compounds; and wherein step c) further comprises recovering at least a portion of said removed quantity F from said removal zone F forming recovered quantity F.

12. The method of claim 11 wherein said reactor feed optionally further comprises at least a portion of said recovered quantity F.

13. The method of claim 1 wherein, following step b), said aqueous stream is passed to a removal zone G comprising a super absorbent polymer for removal of at least a portion of the water from said aqueous stream through contact with said super absorbent polymer and yielding a stream of recovered quantity G comprising concentrated water-soluble organic compounds; and further comprising regenerating said super absorbent polymer by heating at a temperature between about 50° C. and about 90° C. under an inert gas flow having a GHSV of at least about 0.5 h⁻¹.

14. The method of claim 13 wherein said reactor feed optionally further comprises at least a portion of said recovered quantity G.

15. The method of claim 13 wherein, prior to step a), said aqueous stream is passed to a removal zone H comprising a sorbent H selected from the group consisting of zeolite-based adsorbents, clay-based adsorbents, and mixtures thereof, which sorbs at least a portion of the water-soluble organic compounds from said aqueous stream through contact with said sorbent H forming a removed quantity H comprising water-soluble organic compounds; and wherein step c) further comprises recovering at least a portion of said removed quantity H from said removal zone H forming recovered quantity H.

16. The method of claim 15 wherein said reactor feed optionally further comprises at least a portion of said recovered quantity H.

17. A method comprising:
a) passing an aqueous stream comprising a water-soluble complex mixture of organic compounds to a removal zone A for contact with a sorbent A comprising a polymeric microreticular sorbent resin for removal of at least a portion of the water-soluble complex mixture of organic compounds from said aqueous stream forming a removed quantity A comprising water-soluble organic compounds;
b) passing said aqueous stream from said removal zone A to a removal zone B for contact with a sorbent B for removal of at least a portion of the water-soluble complex mixture of organic compounds from said aqueous stream forming a removed quantity B comprising water-soluble organic compounds, wherein said removed quantity B comprises light oxygenated compounds and heavy oxygenated compounds;
c) recovering at least a portion of said removed quantity A from said removal zone A forming recovered quantity A and separating said light oxygenated compounds from said removed quantity B forming recovered quantity B; and
d) charging a reactor feed comprising at least a portion of said recovered quantity B to a basic catalyzed reactor containing a basic catalyst for conversion of said light oxygenated compounds to heavier oxygenated compounds.

18. The method of claim 17 wherein said basic catalyst comprises a material selected from the group consisting of: the oxides, mixed oxides, hydroxides and mixed hydroxides of alkaline metals, alkaline earth metals, Group IIB metals, and Group IIIB metals; mixed oxides between Group IIIA or Group IVA metals with at least one element selected from the group consisting of alkaline metals, alkaline earth metals, Group IIB metals, and Group IIIB metals; mixed hydroxides between Group IIIA or Group IVA metals with at least one element selected from the group consisting of alkaline metals, alkaline earth metals, Group IIB metals, and Group IIIB metals; and mixtures thereof.

19. The method of claim 17 wherein said recovered quantity A comprises light oxygenated compounds and heavy oxygenated compounds; and wherein said reactor feed optionally further comprises said recovered quantity A.

20. The method of claim 17 wherein said light oxygenated compounds comprise compounds selected from the group consisting of ketones, aldehydes, carboxylic acids, and combinations thereof, having 5 or less carbon atoms.

21. The method of claim 17 wherein said light oxygenated compounds are separated from said removed quantity B by thermal desorption comprising:
i) heating said removed quantity B to a temperature in the range of from about 20° C. to about 150° C., under an inert gas stream at up to atmospheric pressure, and partially condensing the resulting first effluent at a temperature in the range of from about 20° C. to about 40° C. for a period of time between about 0.5 to about 4 hours, followed by partial condensation at a temperature in the range of from about −100° C. to about −50° C. for a period of time between about 0.5 to about 4 hours, forming said recovered quantity B comprising said light oxygenated compounds; and
ii) thereafter heating said removed quantity B to a temperature in the range of from about 150° C. to about 500° C. under at least a partial vacuum and for a period of time between about 0.5 to about 4 hours, and partially condensing the resulting second effluent at a temperature in the range of from about −100° C. to about −50° C., forming a second recovered quantity B comprising said heavy oxygenated compounds.

22. The method of claim 17 wherein said light oxygenated compounds are separated from said removed quantity B by chemical displacement using a supercritical solvent selected from the group consisting of supercritical CO₂, supercritical propane, supercritical butane, supercritical toluene, supercritical xylene, and mixtures thereof.

23. A method comprising:
a) separating a bio-oil/water stream comprising a water-soluble complex mixture of organic compounds, water-insoluble organic compounds, and water into a bio-oil stream comprising water-insoluble organic compounds and into an aqueous stream comprising a water-soluble complex mixture of organic compounds;
b) contacting said aqueous stream with an activated carbon-based sorbent for removal of at least a portion of the water-soluble complex mixture of organic compounds from said aqueous stream forming a removed quantity comprising water-soluble organic compounds, wherein the activated carbon-based sorbent has been surface treated in a manner resulting in a reduction in the number of polar and/or charged groups on the surface, and wherein at least 40% of the pore volume of the activated carbon-based sorbent results from pores having diameters in the range of from about 15 Å to about 50 Å; and
c) recovering at least a portion of said removed quantity from said activated carbon-based sorbent forming a recovered quantity comprising light oxygenated compounds and heavy oxygenated compounds; wherein a reactor feed comprising said recovered quantity is charged to a basic catalyzed reactor containing a basic catalyst for conversion of said light oxygenated compounds to heavier oxygenated compounds prior to combination of at least a portion of said recovered quantity with said bio-oil stream.

24. The method of claim 23 wherein said basic catalyst comprises a material selected from the group consisting of: the oxides, mixed oxides, hydroxides and mixed hydroxides of alkaline metals, alkaline earth metals, Group IIB metals, and Group IIIB metals; mixed oxides between Group IIIA or Group IVA metals with at least one element selected from the group consisting of alkaline metals, alkaline earth metals, Group IIB metals, and Group IIIB metals; mixed hydroxides between Group IIIA or Group IVA metals with at least one element selected from the group consisting of alkaline metals, alkaline earth metals, Group IIB metals, and Group IIIB metals; and mixtures thereof.
